(19) United States
(12) Patent Application Publication
(10) Pub. No.: US 2026/0183206 A1
(43) Pub. Date: Jul. 2, 2026

(54) OIL-IN-WATER EMULSION AND ITS USE IN COSMETICS

(71) Applicant: L'OREAL, PARIS (FR)

(72) Inventors: Emmanuelle CAMBLONG, GIGORS ET LOZERON (FR); Thierry COTTON, CHEVILLY-LARUE (FR); Noémie PIOUD, GIGORS ET LOZERON (FR)

(21) Appl. No.: 19/504,922

(22) PCT Filed: Jul. 30, 2024
(86) PCT No.: PCT/EP2024/071511

§ 371 Date: Jan. 23, 2026

(30) Foreign Application Priority Data
Jul. 31, 2023 (FR) 2308274

(51) Int. Cl. A61K 8/06; A61K 8/34; A61K 8/36; A61K 8/55; A61K 8/73; A61K 8/81; A61K 8/9789; A61Q 19/08

(52) U.S. Cl. CPC A61K 8/062; A61K 8/342; A61K 8/345; A61K 8/361; A61K 8/553; A61K 8/735; A61K 8/8158; A61K 8/9789; A61Q 19/08; A61K 2800/34

(57) ABSTRACT

The present invention relates to a composition in the form of an oil-in-water emulsion, which is in particular cosmetic, especially for making up and/or caring for keratin materials, comprising:
at least one fatty phase,
at least one emulsifier comprising at least one phospholipid, and
at least one hydrophilic gelling agent,
   characterized in that the composition comprises less than 2.0% by weight of silicone oil, with respect to the total weight of the composition.

### TECHNICAL FIELD

The present invention relates to the field of caring for and/or making up keratin materials, in particular of the anti-ageing care and/or the hydration of keratin materials, in particular of the skin.

The present invention is targeted at providing a composition for topical application existing in the form of an oil-in-water (O/W) emulsion, and at the cosmetic use of said composition for the care, hygiene, protection and/or make-up of keratin materials, such as the skin of the body or of the face, in particular for the anti-ageing care and/or the hydration of said keratin materials.

The term “keratin materials”, within the meaning of the present invention, is intended in particular to denote the skin, the lips and/or the eyelashes, especially the skin and/or the lips, and preferably the skin of the body and/or the face, and more preferentially of the face.

### PRIOR ART

The formulation of environmentally friendly cosmetic products, that is to say products whose design and development take account of environmental issues, is becoming a major preoccupation for contributing towards meeting the global challenges.

It is thus proving essential to provide more sustainable compositions and/or preparation processes and/or ingredients, thus making it possible to respond to these environmental issues.

In this context, it is important to develop new cosmetic compositions with a better carbon footprint, in particular by promoting the use of starting materials which are renewable and/or which have a good index of naturalness and/or of natural origin and more particularly of plant origin, while reducing the use of compounds of petrochemical origin.

Still in this context, new emulsifying systems capable of meeting both this environmental requirement and exhibiting appropriate properties as regards sensory feelings and stability are constantly being sought.

An emulsifying system comprising at least one phospholipid, in particular at least hydrogenated lecithin, satisfies the criterion of environmental friendliness. In particular, it has been reported that this emulsifier of natural origin makes possible the formation of lamellar O/W emulsions offering perfect affinity with the skin, with a velvety, soft and gliding feel.

Nevertheless, the formulation of compositions for the anti-ageing care and/or the hydration of the skin, employing such an emulsifying system, can prove to be difficult in terms of stability. This is because it has been possible to observe the release of aqueous exudates in compositions in oil-in-water emulsion form which have been formulated from such an emulsifier.

### DISCLOSURE OF THE INVENTION

There thus exists a need for eco-designed cosmetic compositions which are improved in terms of naturalness, biodegradability, durability and renewability without compromise in sensoriality, in the context of the development of compositions for the anti-ageing care and/or for the hydration of the skin.

In particular, there remains a need to formulate such compositions exhibiting a sensoriality adapted to the needs of anti-ageing and/or moisturizing compositions and exhibiting good stability.

Finally, there also remains a need to have available compositions which are compatible with the current requirements of consumers, in particular from an environmental viewpoint.

The present invention is targeted specifically at meeting all or some of these needs.

### SUMMARY OF THE INVENTION

Thus, according to a first of its aspects, the present invention relates to a composition in the form of an oil-in-water emulsion, which is in particular cosmetic, especially for making up and/or caring for keratin materials, comprising:
at least one fatty phase,
at least one emulsifier comprising at least one phospholipid, and
at least one hydrophilic gelling agent,
   characterized in that the composition comprises less than 2.0% by weight of silicone oil, with respect to the total weight of the composition.

This is because the inventors have found, surprisingly, that the formulation of a combination of an emulsifying system comprising at least one phospholipid, in particular at least hydrogenated lecithin, and of at least one hydrophilic gelling agent, in particular chosen from polymers and copolymers of 2-acrylamido-2-methylpropanesulfonic acid, which are optionally crosslinked and/or neutralized, such as poly(2-acrylamido-2-methylpropanesulfonic acid), in a composition in the form of an oil-in-water emulsion, exhibiting a content of less than 2.0% by weight of silicone oil, with respect to the total weight of the composition, advantageously makes it possible to obtain a composition reconciling both excellent stability and excellent sensory performance qualities, in particular a reduction in whitening on application and a reduction in, indeed even an absence of, whitening after application to the skin.

In addition, the compositions thus obtained exhibit good rheological properties, in particular in terms of viscosity and/or of texture, and cosmetic properties which are particularly advantageous, especially in terms of reduced soaping effects during application and after application and a very good anti-ageing and/or moisturizing effect.

In particular, the compositions obtained exhibit a satisfactory appearance and a flexible texture, and are easy to apply and to spread. After application, such compositions leave a feeling of freshness, of hydration, of nutrition, of softness and of comfort on the skin. Moreover, no greasy, oily or sticky effect on application of these compositions are obtained and no sensation of irritation or of discomfort is present.

Finally, the compositions obtained exhibit a maintenance of the effects obtained from the time of application, in particular throughout the day, during which a sensation of comfort and freshness remains present. The skin also remains hydrated and soft, it breathes and is not choked, or greasy or sticky or shiny, and tightness sensations, dryness, desquamation and roughness are prevented.

Advantageously, a composition according to the invention is more natural while meeting the requirements expected by users in the field of anti-ageing or moisturizing compositions. According to one embodiment, the composition according to the invention comprises less than 1.0% by weight of silicone oil(s), preferably less than 0.5% by weight of silicone oil(s), with respect to the total weight of the composition, and more preferentially is devoid of silicone oil(s).

A composition according to the invention is in particular employed for caring for and/or making up keratin materials, and preferably for caring for keratin materials.

Thus, the invention also relates, according to another of these aspects, to a cosmetic process for making up and/or caring for, preferably caring for, keratin materials, in particular the skin, comprising at least a stage of application, to said keratin materials, of a composition according to the invention.

In particular, a composition according to the invention can be employed with the aim of combating signs of skin ageing, in particular skin photo-ageing, or alternatively for the purpose of moisturizing the skin.

Thus, the present patent application also relates to the cosmetic use of a composition according to the invention in order to combat signs of skin ageing, in particular skin photo-ageing, or in order to moisturize the skin.

It is understood that the processes and methods defined according to the present invention are non-therapeutic.

Other characteristics, variants and advantages of the compositions according to the invention will become more clearly apparent on reading the description and the examples which will follow.

### DETAILED DESCRIPTION

### Composition

As stated above, a composition according to the invention can be cosmetic and/or dermatological, and is preferably cosmetic.

A composition according to the invention is generally suitable for topical application to the skin and thus generally comprises a physiologically acceptable medium, that is to say a medium compatible with the skin.

It is preferably a cosmetically acceptable medium, that is to say a medium which exhibits a pleasant colour, odour and feel and which does not cause unacceptable discomfort, that is to say stinging, tightness or redness, liable to dissuade the user from applying this composition.

A cosmetic composition according to the invention is provided in the form of a direct or oil-in-water emulsion, namely an emulsion comprising a continuous aqueous phase and a fatty phase, in particular in a content of at least 8% by weight, dispersed in said aqueous phase.

A composition according to the invention can be prepared according to techniques well known to a person skilled in the art.

### Phospholipid

The composition according to the invention comprises at least one phospholipid.

The phospholipid is preferably chosen from:
natural phospholipids, in particular egg or soya lecithin, or sphingomyelin,
chemically or enzymatically modified phospholipids, in particular hydrogenated lecithin, and
synthetic phospholipids, in particular dipalmitoylphosphatidylcholine.

The phospholipid is preferably hydrogenated lecithin.

Preferably, the phospholipid is present in a mixture with fatty alcohols, such as alcohols having from 12 to 16 carbon atoms, and/or with a fatty acid, such as palmitic acid.

Preferentially, the phospholipid is sold under the INCI name C12-16 Alcohols (and) Palmitic Acid (and) Hydrogenated Lecithin and in particular under the reference Biophilic™ H MB from Lucas Meyer Cosmetics.

The phospholipid is present in the composition according to the invention in an amount preferably of between 0.01% and 3% by weight, more preferentially between 0.05% and 2% by weight and better still between 0.1% and 1% by weight, with respect to the total weight of the composition.

### Hydrophilic Gelling Agent

As already mentioned above, the composition according to the invention comprises at least one hydrophilic gelling agent.

The hydrophilic gelling agent according to the invention can be synthetic or natural.

The hydrophilic gelling agent (or thickener) can in particular be chosen from:

### 1) Carboxyvinyl Polymers

The carboxyvinyl polymers may or may not be modified.

They can be salts of polyacrylates. These are in particular crosslinked and neutralized polymers, in non-particulate form.

The sodium salts are advantageously preferred.

Mention may in particular be made of polymers bearing the INCI name Sodium Polyacrylate, such as:
Cosmedia SP® or crosslinked sodium polyacrylate containing 90% solids and 10% water, Cosmedia SPL® or sodium polyacrylate as an inverse emulsion containing approximately 60% dry active matter, an oil (hydrogenated polydecene) and a surfactant (PPG-5 Laureth-5), both sold by BASF; and
partially neutralized crosslinked sodium polyacrylates in the form of an inverse emulsion comprising at least one polar oil, for example that sold under the name Luvigel® EM sold by BASF or under the name Salcare AST from CIBA (BASF) (INCI name: Sodium Acrylates Copolymer (and) PPG-1 Trideceth-6 (and) Glycine soja (Soybean) Oil).

Mention may also be made, in particular, of the polymers bearing the INCI name Sodium Polymethacrylate, for example Darvan® 7-N from Vanderbilt.

Mention may also be made, among the carboxyvinyl polymers suitable for the invention, of those sold under the following names:
Carbopol® (INCI name: Carbomer) from Goodrich;
Pemulen™ (INCI name: Acrylates/C10-30 Alkyl Acrylate Crosspolymer) from Goodrich;
Aqupec MG N40R (INCI name: Sodium Carbomer) from Sumitomo Seika;
Aculyn™ 88 Polymer (INCI name: Acrylates/Steareth-20 Methacrylate Crosspolymer) from Dow Chemical;
Aculyn™ 28 Polymer (INCI name: Acrylates/Beheneth-25 Methacrylate Copolymer) from Dow Chemical;
Aristoflex® Velvet from Clariant (INCI: Polyacrylate Crosspolymer-11).

The carboxyvinyl polymers can be present in the composition according to the invention in an amount of dry matter comprises between 0.1% and 3% by weight, more particularly between 0.1% and 2% by weight, with respect to the total weight of the composition.

2) polymers and copolymers of 2-acrylamido-2-methylpropanesulfonic acid (AMPS), in particular homopolymers or copolymers, which are crosslinked or non-crosslinked, comprising at least the 2-acrylamido-2-methylpropanesulfonic acid (AMPS) monomer, in particular in the form partially or completely neutralized by an inorganic base other than aqueous ammonia, such as sodium hydroxide or potassium hydroxide.

These AMPS polymers can be completely or virtually completely neutralized, that is to say neutralized to at least 90%.

These AMPS polymers can be crosslinked or non-crosslinked.

The AMPS polymers suitable for the invention are hydrophilic, that is to say water-soluble or water-dispersible. They can be:
either “homopolymers” comprising only AMPS monomers and, if they are crosslinked, one or more crosslinking agents, such as those defined below;
or copolymers obtained from AMPS and from one or more hydrophilic or hydrophobic monomers, for example ethylenically unsaturated monomers, and, if they are crosslinked, one or more crosslinking agents, such as those defined below. When said copolymers comprise hydrophobic ethylenically unsaturated monomers, are preferably present in small amounts.

The term “water-soluble or water-dispersible” is understood to mean polymers which, for example, introduced into an aqueous phase at 25° C., at a concentration by weight equal to 1%, make it possible to obtain a macroscopically homogeneous and transparent solution, in particular having a maximum light transmittance value, at a wavelength equal to 500 nm, through a sample of 1 cm in thickness, of at least 60%, preferably of at least 70%.

The polymers and copolymers of 2-acrylamido-2-methylpropanesulfonic acid (AMPS), which are optionally crosslinked and/or neutralized, are, for example, chosen from:
a) poly(2-acrylamido-2-methylpropanesulfonic acid) polymers, in particular the homopolymers, polyAMPSs, which are crosslinked and at least 90% neutralized.

They are generally characterized in that they comprise, randomly distributed:
i) from 90% to 99.9% by weight of units of following general formula (1):

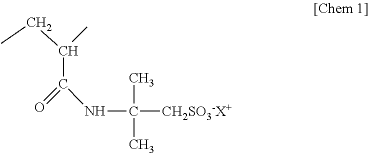

in which X⁺ denotes a cation or a mixture of cations, it being possible for at most 10 molar % of the cations X⁺ to be protons H⁺; and
ii) from 0.01% to 10% by weight of crosslinking units originating from at least one monomer having at least two olefinic double bonds, the proportions by weight being defined with respect to the total weight of the polymer.

This polymer preferably comprises from 98% to 99.5% by weight of units of formula (1) and from 0.2% to 2% by weight of crosslinking units.

The cation X⁺ represents a cation or a mixture of cations chosen in particular from a proton, an alkali metal cation, a cation equivalent to that of an alkaline earth metal or the ammonium ion. The preferential cation X⁺ is the NH₄⁺ cation. More particularly, from 90 mol % to 100 mol % of the cations are NH₄⁺ cations and from 0 mol % to 10 mol % are protons (H⁺).

The crosslinking monomers having at least two olefinic double bonds are chosen, for example, from dipropylene glycol diallyl ether, polyglycol diallyl ethers, triethylene glycol divinyl ether, hydroquinone diallyl ether, tetraallyloxyethane or other polyfunctional alcohol allyl or vinyl ethers, tetraethylene glycol diacrylate, triallylamine, trimethylolpropane diallyl ether, methylenebisacrylamide or divinylbenzene.

The crosslinking monomers bearing at least two olefinic double bonds are more particularly chosen from those corresponding to the following general formula (2):

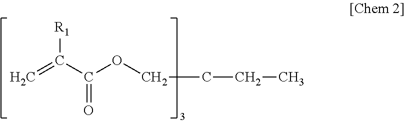

in which R₁ denotes a hydrogen atom or a C₁-C₄ alkyl radical and more particularly the methyl radical (trimethylolpropane triacrylate).

Mention may in particular be made, as polymers of this type, of crosslinked or non-crosslinked polymers of sodium 2-acrylamido-2-methylpropanesulfonate, such as that used in the commercial product Simulgel™ 800 (INCI name: Sodium Polyacryloyldimethyl Taurate (and) Polysorbate 80 (and) Sorbitan Oleate) from Seppic, crosslinked polymers of ammonium 2-acrylamido-2-methylpropanesulfonate (INCI name: Ammonium Polyacryloyldimethyl Taurate), such as sold under the name Hostacerin® AMPS by Clariant. In particular, mention may be made, as AMPS homopolymers suitable for the invention, of the crosslinked and neutralized homopolymer of 2-acrylamido-2-methylpropanesulfonic acid sold by Clariant under the trade name Hostacerin® AMPS (INCI name: Ammonium Polyacryloyldimethyl Taurate).
b) AMPS copolymers, containing in particular water-soluble ethylenically unsaturated monomers, hydrophobic monomers or their mixtures.

The water-soluble comonomers can be ionic or non-ionic.

Mention may be made, among the ionic water-soluble comonomers, for example, of the following compounds and their salts:
(meth)acrylic acid,
styrenesulfonic acid,
vinylsulfonic acid and (meth)allylsulfonic acid,
vinylphosphonic acid,
maleic acid,
itaconic acid,
crotonic acid,
the water-soluble vinyl monomers of following formula (3):

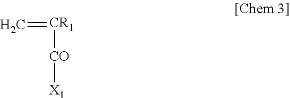

in which:
R₁ is chosen from H, —CH₃, —C₂H₅ or —C₃H₇;
X₁ is chosen from alkyl oxides of —OR₂ type where R₂ is a saturated or unsaturated, linear or branched, hydrocarbon radical having from 1 to 6 carbon atoms, substituted by at least one sulfonic (—SO₃—) and/or sulfate (—SO₄—) and/or phosphate (—PO₄H₂—) group.

Mention may be made, among the non-ionic water-soluble comonomers, for example, of:
(meth)acrylamide,
N-vinylacetamide and N-methyl-N-vinylacetamide,
N-vinylformamide and N-methyl-N-vinylformamide,
maleic anhydride,
vinylamine,
N-vinyllactams comprising a cyclic alkyl group having from 4 to 9 carbon atoms, such as N-vinylpyrrolidone, N-vinylbutyrolactam and N-vinylcaprolactam,
vinyl alcohol of formula CH₂═CHOH,
the water-soluble vinyl monomers of following formula (4):

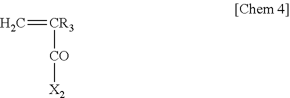

in which:
R₃ is chosen from H, —CH₃, —C₂H₅ or —C₃H₇;
X₂ is chosen from alkyl oxides of —OR₄ type where R₄ is a saturated or unsaturated, linear or branched, hydrocarbon radical having from 1 to 6 carbons, optionally substituted by a halogen atom (iodine, bromine, chlorine, fluorine) or a hydroxyl (—OH) or ether group.

Mention may be made, for example, of glycidyl (meth)acrylate, hydroxyethyl methacrylate, and ethylene glycol, diethylene glycol or polyalkylene glycol (meth)acrylates.

Mention may be made, among the hydrophobic comonomers without a fatty chain, for example, of:
styrene and its derivatives, such as 4-butylstyrene, α-methylstyrene and vinyltoluene;
vinyl acetate of formula CH₂═CH—OCOCH₃;
vinyl ethers of formula CH₂═CHOR in which R is a saturated or unsaturated, linear or branched, hydrocarbon radical having from 1 to 6 carbons;
acrylonitrile;
caprolactone;
vinyl chloride and vinylidene chloride;
silicone derivatives, resulting after polymerization in silicone polymers, such as methacryloyloxypropyltris(trimethylsiloxy)silane and silicone methacrylamides;
hydrophobic vinyl monomers of following formula (5):

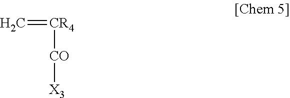

in which:
R₄ is chosen from H, —CH₃, —C₂H₅ or —C₃H₇;
X₃ is chosen from alkyl oxides of —OR₅ type where R₅ is a saturated or unsaturated, linear or branched, hydrocarbon radical having from 1 to 6 carbon atoms.

Mention may be made, for example, of methyl methacrylate, ethyl methacrylate, n-butyl (meth)acrylate, tert-butyl (meth)acrylate, cyclohexyl acrylate, isobornyl acrylate and 2-ethylhexyl acrylate.

The AMPS copolymers can be, in particular:
i) copolymers of AMPS and of acrylamide, more particularly crosslinked anionic copolymers of AMPS and of acrylamide.

These copolymers can in particular be crosslinked by a polyolefinically unsaturated compound, such as those chosen from the group constituted of tetraallyloxyethane, allylpentaerythritol, methylenebisacrylamide, allyl sucrose and pentaerythritol.

Preferentially, use is made of methylenebisacrylamide, partially or completely neutralized by a neutralizing agent, such as sodium hydroxide, potassium hydroxide, aqueous ammonia or an amine, such as triethanolamine.

Preferably, said polyolefinically unsaturated compound is present in the copolymer at a concentration of between 0.06 and 1 mmol per mole of the mixture of monomers.

The preferred copolymers are obtained by radical copolymerization of 15-85 mol % of acrylamide and of 15-85 mol % of 2-acrylamido-2-methylpropanesulfonic acid, in particular of 30-70 mol % of acrylamide and of 30-70 mol % of 2-acrylamido-2-methylpropanesulfonic acid, and better still of 55-70 mol % of acrylamide and of 30-45 mol % of 2-acrylamido-2-methylpropanesulfonic acid.

Moreover, the 2-acrylamido-2-methylpropanesulfonic acid can be generally at least partially neutralized in the form of a salt, for example by sodium hydroxide, by potassium hydroxide or by a low-molecular-weight amine, such as triethanolamine, or their mixtures.

The hydrophilic gelling agent capable of being employed can also be chosen from crosslinked anionic copolymers of acrylamide and of 2-acrylamido-2-methylpropanesulfonic acid.

A crosslinked copolymer which is particularly preferred in the context of the implementation of the present invention is in particular available under the name Sepigel 305™ sold by Seppic (C.T.F.A. name Polyacrylamide/C13-14 Isoparaffin/Laureth 7) or Aquagel 65 or Aquagel 35 (INCI name: Polyacrylamide (and) C9-11 Pareth-6) from Tinci Materials; mention may also be made of the product Simulgel™ 600 (C.T.F.A. name Acrylamide/Sodium Acryloyldimethyl Taurate Copolymer/Isohexadecane/Polysorbate 80) sold by Seppic.
ii) copolymers derived from 2-acrylamido-2-methylpropanesulfonic acid (AMPS) which are or are not crosslinked, comprising at least in particular one hydrophobic group comprising:
   from 80 to 99 mol % of 2-acrylamido-2-methylpropanesulfonic acid (AMPS) unit of following formula (6):

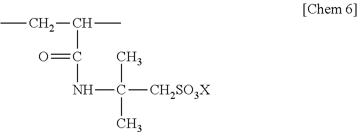

in which X is a proton, an alkali metal cation, an alkaline earth metal cation or the ammonium ion, it being understood that, when X is an alkaline earth metal cation, it shares 2 positive charges with 2 SO₃ groups; and
from 1 mol % to 20 mol % and preferably from 1 mol % to 15 mol % of unit of following formula (7):

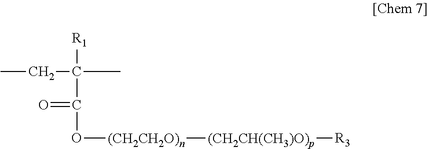

in which n and p, independently of each other, denote a number of moles and varies from 0 to 30, preferably from 1 to 20, provided that n+p is less than or equal to 30, preferably less than 25 and better still less than 20, R₁ denotes a hydrogen atom or a linear or branched C₁-C₆ alkyl (preferably methyl) radical and R₃ denotes a linear or branched alkyl group comprising m carbon atoms ranging from 6 to 30, preferably from 10 to 25, carbon atoms. These polymers are preferentially partially or completely neutralized by an inorganic base, such as, for example, sodium hydroxide, potassium hydroxide or aqueous ammonia, or by an organic base, such as mono-, di- and triethanolamine, aminomethylpropanediol, N-methylglucamine, basic amino acids, such as arginine and lysine, and their mixtures.

When the polymer is crosslinked, the crosslinking agents can be chosen from the polyolefinically unsaturated compounds commonly used for the crosslinking of polymers obtained by radical polymerization.

Mention may be made, for example, as crosslinking agents, of divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric and/or vinylphosphonic acid derivatives, or mixtures of these compounds.

According to one embodiment, the crosslinking agent is chosen from methylenebisacrylamide, allyl methacrylate or trimethylolpropane triacrylate (TMPTA). The degree of crosslinking generally ranges from 0.01 mol % to 10 mol % and more particularly from 0.2 mol % to 2 mol % with respect to the polymer.

Mention may be made, as polymers derived from AMPS which can be used in the composition according to the invention, of polymers prepared from 2-acrylamido-2-methylpropanesulfonic acid (AMPS) or one of its sodium or ammonium salts with an ester of (meth)acrylic acid and of an oxyethylenated C₁₀ to C₂₀ alcohol comprising from 6 to 25 oxyethylenated groups.

According to one embodiment, the polymer derived from AMPS is a copolymer of AMPS and of a C₁₆-C₁₈ alkyl methacrylate comprising from 6 to 25 oxyethylenated groups, obtained from methacrylic acid or from a salt of methacrylic acid and from a C₁₆-C₁₈ alcohol oxyethylenated by 6 to 25 mol of ethylene oxide. The amphiphilic polymer can also be a copolymer of AMPS and of a C₁₂-C₁₄ alkyl methacrylate comprising from 6 to 25 oxyethylenated groups, obtained from methacrylic acid or from a salt of methacrylic acid and from a C₁₂-C₁₄ alcohol oxyethylenated by 6 to 25 mol of ethylene oxide.

Mention may in particular be made of:
the non-crosslinked copolymer obtained from 92.65 mol % of AMPS and from 7.35 mol % of a C₁₆-C₁₈ alcohol methacrylate comprising 8 oxyethylenated groups (Genapol T-080), such as that sold by Clariant under the name Aristoflex® SNC,
the non-crosslinked copolymer obtained from 91.5 mol % of AMPS and from 8.5 mol % of a C₁₂-C₁₄ alcohol methacrylate comprising 7 oxyethylenated groups (Genapol LA-070), such as that sold by Clariant under the name Aristoflex® LNC, and
their mixtures.

Mention may more especially be made, as crosslinked AMPS polymers of this type, of that sold under the name Aristoflex® HMS by Clariant, which is a crosslinked AMPS/ethoxylated (25 EO) stearyl methacrylate copolymer, or that sold under the name Aristoflex® HMB by Clariant, which is a crosslinked AMPS/ethoxylated (25 EO) behenyl methacrylate copolymer. A mixture of these polymers can also be used.

Mention may also be made, as other polymers derived from AMPS, in particular AMPS copolymers, which can be used in the composition according to the invention, of:
copolymers of AMPS and of vinylpyrrolidone or of vinylformamide, such as those used in commercial products sold by Clariant under the name Aristoflex® AVC (INCI: Ammonium Acryloyldimethyl Taurate/VP Copolymer), Aristoflex® AVS (INCI: Sodium Acryloyldimethyl Taurate/VP Crosspolymer) or Aristoflex® AVL from Clariant (INCI: Caprylic/Capric Triglyceride (and) Ammonium Acryloyldimethyl Taurate/VP Copolymer (and) Trilaureth-4 Phosphate (and) Polyglyceryl-2 Sesquiisostearate), for example neutralized by sodium hydroxide or potassium hydroxide;
copolymers of AMPS and of sodium acrylate, such as, for example, the AMPS/sodium acrylate copolymer, such as that used in the commercial product sold under the name Simulgel™ EG (INCI name: Sodium Acrylate/Sodium Acryloyldimethyl Taurate Copolymer (and) Isohexadecane (and) Polysorbate 80) by Seppic or under the trade name Sepinov™ EMT 10 by Seppic (CTFA name: Hydroxyethyl Acrylate/Sodium Acryloyldimethyl Taurate Copolymer);
copolymers of AMPS and of hydroxyethyl acrylate, such as, for example, the AMPS/hydroxyethyl acrylate copolymer, such as that used in the commercial product sold under the name Simulgel™ NS by Seppic (INCI name: Hydroxyethyl Acrylate/Sodium Acryloyldimethyl Taurate Copolymer and Squalane and Polysorbate 60) or like the commercial product sold under the name Sepinov™ EMT 10 (INCI name: Hydroxyethyl Acrylate/Sodium Acryloyldimethyl Taurate Copolymer) by Seppic; and
copolymers of AMPS and of biopolymer, such as copolymers of AMPS and of polysaccharide, such as, for example, the commercial product sold under the name Aristoflex® Eco T by Clariant (INCI name: Caesalpinia Spinosa Gum/Ammonium AMPS Crosspolymer).

The AMPS polymers and copolymers can be present in the composition according to the invention in an amount of dry matter comprises between 0.1% and 10% by weight, in particular between 0.1% and 2.5% by weight, more particularly between 0.5% and 1.5% by weight, with respect to the total weight of the composition.

The AMPS polymers have, for example, a molar mass ranging from 50 000 g/mol to 10 000 000 g/mol, in particular from 80 000 g/mol to 8 000 000 g/mol, more particularly from 100 000 g/mol to 7 000 000 g/mol.

### 3) Heterogeneous Polyholosides

The term “heterogeneous polyholoside” is understood to mean, according to the present invention, polymers constituted of the combination of different monosaccharides or of monosaccharides having the same empirical chemical formula but of different geometrical configuration (D and L isomers, for example).

These polymers differ both from polyheterosides, which are constituted of one or more monosaccharides and of a non-carbohydrate part, and from homogeneous polyholosides, which result from the combination of one and the same monosaccharide.

Thus, the heterogeneous polyholoside according to the invention is constituted solely of monosaccharides and results from the combination of at least two different monosaccharides.

The polyholosides according to the invention can be constituted of 2 to 10 monosaccharides, compounds commonly known as oligoholosides, or of more than 10 monosaccharides, compounds commonly known as polyholosides.

The monosaccharides present in the polyholoside according to the invention can be chosen from all the envisageable monosaccharides, of natural or synthetic origin, and in particular such as:
i) aldoses, such as:
   pentoses: ribose, arabinose, xylose or apiose, for example,
   hexoses: glucose, fucose, mannose or galactose, for example,
ii) ketoses, such as fructose,
iii) deoxymonosaccharides, such as rhamnose, digitoxose, cymarose or oleandrose,
iv) monosaccharide derivatives, such as uronic acids, like mannuronic acid, guluronic acid, galacturonic acid or glycuronic acid, or also itols, like mannitol or sorbitol.

The polyholoside according to the invention can be branched or linear. It can also be substituted, for example by fatty chains, in particular comprising from 8 to 30 carbon atoms. Moreover, the polyholoside according to the invention can be an alginate (poly mannuronate and guluronate), such as a sodium alginate, a propylene glycol alginate, a calcium alginate or a glyceryl alginate.

However, the heterogeneous polyholoside preferably comprises at least one fucose unit, which can be present in an amount of 10-90% by weight, preferably 15-35% by weight, with respect to the weight of polyholoside under dry matter.

In particular, the polyholoside according to the invention can comprise fucose, galactose and galacturonic acid units, and, for example, can comprise a linear sequence of α-L-fucose, of α-D-galactose and of galacturonic acid. In this case, it preferably exhibits a viscosity of 800-1200 mPa·s (Brookfield LV31 viscosity, 12 revolutions/min, at 30° C.) when it is dissolved in water at a concentration of approximately 1% by weight. Such a polyholoside is in particular available in the form of a 1% solution in water from Solabia under the trade name Fucogel 1000 PP® (INCI name: Biosaccharide Gum-1) or in the form of a 1.1% solution in water from Solabia under the trade name Fucocert (INCI name: Biosaccharide Gum-1 (and) Sodium Levulinate (and) Glyceryl Caprylate (and) Sodium Anisate).

The polyholosides according to the invention are preferably introduced into the composition in the form of an aqueous solution which can comprise from 0.1% to 5% by weight of polyholoside.

The polyholosides can be present in the composition according to the invention in an amount of dry matter ranging more particularly from 0.1% to 10%, preferably from 0.1% to 2%, by weight, with respect to the total weight of the composition.

### 4) Glyceryl Acrylate Polymers

These glyceryl acrylate polymers are chosen in particular from copolymers of glyceryl acrylate and of acrylic acid. Such copolymers are sold in particular under the names Lubrajel® MS, Lubrajel® CG, Lubrajel® DV, Lubrajel® NP, Lubrajel®L Oil, Lubrajel® Oil BG, Lubrajel® PF, Lubrajel® TW and Lubrajel® WA by Guardian Laboratories or Ashland. Preferably, Lubrajel® MS is used.

The glyceryl acrylate polymers can be present in the composition according to the invention in an amount of dry matter ranging more particularly from 0.1% to 10% by weight, preferably from 0.1% to 2% by weight with respect to the total weight of the composition.

5) Polysaccharides, in particular polysaccharides produced by microorganisms, polysaccharides isolated from algae and polysaccharides of higher plants.

The polysaccharides according to the invention can be chosen from:
algal extracts, such as alginates, carrageenans, agar-agar and their mixtures. Mention may be made, as examples of carrageenans, of kappa-carrageenan, and in particular of the compounds sold under the names Satiagum UTC30® and Satiagum UTC10® from Cargill; mention may be made, as alginates, of sodium alginate sold under the name Kelcosol® by ISP, under the name Sobalg PH 460 by Danisco or under the name Flavikafine™ S by Nisshinbo Chemical;
gums, such as xanthan gums, guar gums and their non-ionic derivatives (hydroxypropyl guar), gums arabic, konjac gums, mannan gums, gums tragacanth, ghatti gums, karaya gums, locust bean gums, biopolysaccharide gums of microbial origin, such as scleroglucan or xanthan gums; mention may be made, as examples, of the guar gum sold under the name Jaguar HP 105® by Solvay; the konjac mannan Gum® (1% glucomannan) sold by GfN; the diutan gum Kelco-Care™ sold by Lubrizol (INCI name: Sphingomonas Ferment Extract); the gum Solagum™ TARA 38553F sold by Seppic (INCI name: Caesalpinia spinosa Gum); the gellan gum Kelcogel® CG LA sold by CP Kelco (INCI name: Gellan Gum);
modified or unmodified starches, such as those resulting, for example, from cereals, such as wheat, maize or rice, from vegetables, such as yellow split peas, from tubers, such as potatoes or cassava, tapioca starches; dextrins, such as maize dextrins; mention may in particular be made, as examples, of the rice starch Remy DR I® (INCI name: Oryza sativa (Rice) Starch) sold by Remy; the maize starch B® sold by Roquette (INCI name: Zea mays (Corn) Starch); the modified maize starch Dry-Flo® AF sold by AkzoNobel (Nouryon) (INCI name: Corn Starch Modified); the potato starch Starkina™ Natural sold by Agrana Starke (INCI name: Solanum tuberosum Starch); the potato starch modified by 2-chloroethyl aminodipropionic acid neutralized with sodium hydroxide sold under the name Structure Solanace® by Nouryon; the native tapioca starch powder sold under the name Tapioca Pure® by Nouryon; the starch StarDesign™ Power sold by Cargill (INCI name: Sodium Starch Octenylsuccinate (and) Hydroxypropyl Starch Phosphate); the dextrin extracted from maize under the name Index® from National Starch;
pectic acids and pectins, such as the pectin sold under the name GENU pHresh™ by CP Kelco (INCI name: Pectin);
celluloses and their derivatives, in particular alkyl- or hydroxyalkyl celluloses; mention may in particular be made of methyl celluloses, hydroxyalkyl celluloses, ethylhydroxyethyl celluloses or carboxymethyl celluloses. Mention may be made, as examples, of cetyl hydroxyethyl celluloses under the names Polysurf 67CS® and Natrosol Plus 330® from Ashland; methyl hydroxyethyl celluloses under the names Tylose® MH 300 and Tylose® MH 1000 from Sigma-Aldrich (INCI: Methyl Hydroxyethylcellulose); celluloses and cellulose gums under the name Betafib ETD from DKSH (INCI name: Cellulose (and) Cellulose Gum); cellulose gums and microcrystalline celluloses under the names Natpure® Cellgum Plus from Sensient or Avicel® PC 611 from DuPont (INCI: Microcrystalline Cellulose (and) Cellulose Gum);
fructans, glucans, amylose, amylopectin, glycogen, pullulan, dextrans, mannans, xylans, lignins, arabans, galactans, galacturonans, chitin, chitosans, glucuronoxylans, arabinoxylans, xyloglucans, glucomannans, arabinogalactans, mucopolysaccharides (glycosaminoglycans), such as chondroitin sulfates;
and their mixtures.

The polysaccharides can be present in the composition according to the invention in an amount of dry matter comprises between 0.01% and 2% by weight, in particular between 0.05% and 1% by weight and more particularly still between 0.05% and 0.8% by weight, with respect to the total weight of the composition.

Use is preferably made of the polysaccharides chosen from gums, such as xanthan gum, and a carrageenan. Preferably, use is made of the polysaccharides chosen from xanthan gum and a carrageenan.

Xanthan gums have in particular a molecular weight of between 1 000 000 g/mol and 50 000 000 g/mol and a viscosity of between 0.6 and 1.65 Pa·s for an aqueous composition containing 1% of xanthan gum (measured at 25° C. on a Brookfield viscometer of LVT type at 60 revolutions per minute).

The xanthan gums are represented, for example, by the products sold under the names Rhodicare by Rhodia Chimie, under the name Satiaxane™ by Cargill Texturizing, under the name Novaxan™ by ADM and under the names Kelzan® and Keltrol® by CP Kelco.

6) Clays, modified or unmodified, which can be present in a content as active material of between 0.05% and 10% by weight and preferentially between 0.1% and 5% by weight, with respect to the total weight of the composition.

The composition according to the invention can comprise a mixture of the various hydrophilic gelling agents mentioned above.

Thus, according to a particular embodiment, a composition according to the invention can comprise a hydrophilic gelling agent chosen from:
carboxyvinyl polymers,
polymers and copolymers of 2-acrylamido-2-methylpropanesulfonic acid, which are optionally crosslinked and/or neutralized, in particular chosen from (a) poly(2-acrylamido-2-methylpropanesulfonic acid) polymers, more particularly the homopolymers, polyAMPSs, which are crosslinked and at least 90% neutralized, and (b) AMPS copolymers, more particularly chosen from (i) copolymers of AMPS and of acrylamide, for example crosslinked anionic copolymers of acrylamide and of AMPS, (ii) copolymers derived from 2-acrylamido-2-methylpropanesulfonic acid which are or are not crosslinked, comprising at least one hydrophobic group, (iii) copolymers of AMPS and of vinylpyrrolidone or of vinylformamide, (iv) copolymers of AMPS and of sodium acrylate, (v) copolymers of AMPS and of hydroxyethyl acrylate, and (vi) copolymers of AMPS and of biopolymer;
heterogeneous polyholosides,
glyceryl acrylate polymers,
polysaccharides,
clays, and
their mixtures.

According to another particular embodiment, the hydrophilic gelling agent is chosen from polymers and copolymers of 2-acrylamido-2-methylpropanesulfonic acid, such as the crosslinked and neutralized homopolymer of 2-acrylamido-2-methylpropanesulfonic acid, sold in particular by Clariant under the trade name Hostacerin® AMPS (INCI name: Ammonium Polyacryloyldimethyl Taurate).

Said hydrophilic gelling agent chosen from polymers and copolymers of 2-acrylamido-2-methylpropanesulfonic acid can be present in a content of between 0.1% and 10% by weight, in particular between 0.1% and 2.5% by weight, more particularly between 0.5% and 1.5% by weight, with respect to the total weight of the composition.

According to another particular embodiment, the composition according to the invention comprises at least one hydrophilic gelling agent chosen from polymers and copolymers of 2-acrylamido-2-methylpropanesulfonic acid, in particular a crosslinked and neutralized homopolymer of 2-acrylamido-2-methylpropanesulfonic acid, and at least one additional gelling agent, in particular chosen from polysaccharides, in particular as are described above, more particularly xanthan gum or a carrageenan.

It has in particular been observed that, in the presence of xanthan gum, the composition in the form of an oil-in-water emulsion according to the invention can be refined as illustrated in Example 3. In other words, in this Example 3, the microscopic appearance of a composition in the form of an oil-in-water emulsion according to the invention comprising xanthan gum is advantageously finer, in comparison with a composition in the form of an oil-in-water emulsion according to the invention not comprising xanthan gum.

Said additional gelling agent can be present in a content of between 0.01% and 2% by weight, in particular between 0.05% and 1% by weight and more particularly still between 0.05% and 0.8% by weight, with respect to the total weight of the composition.

### Aqueous Phase

As mentioned above, a composition according to the invention comprises at least one physiologically acceptable aqueous phase. The term “physiologically acceptable” is understood to mean a medium compatible with keratin materials.

The composition according to the invention is an oil-in-water (O/W) emulsion. The aqueous phase is the continuous phase.

The composition according to the invention preferably comprises an aqueous phase comprising water and optionally one or more polyol(s). This (these) polyol(s) can advantageously be chosen, for example, from polyols having from 2 to 20 carbon atoms, preferably from 2 to 6 carbon atoms, such as glycerol, diglycerol, propylene glycol, isoprene glycol, dipropylene glycol, butylene glycol, hexylene glycol, 1,3-propanediol, pentylene glycol, polyethylene glycols having from 2 to 200 ethylene oxide units and their mixtures.

According to a particular embodiment, the aqueous phase comprises water and at least one polyol having in particular from 2 to 20 carbon atoms.

According to a particular embodiment, the aqueous phase comprises at least one alkanol.

The term “alkanol” is understood to mean linear or branched C₂-C₄ alkanols, such as ethanol (otherwise described as denatured alcohol in the examples which will follow), isopropanol, propanol or butanol.

According to one embodiment, the aqueous phase is present in a content of between 65% and 95% by weight, preferably between 70% and 92% by weight and more preferentially between 74% and 82% or 88% and 92% by weight, with respect to the total weight of the composition.

The amount of polyols can range, for example, from 0.1% to 20% by weight, preferably from 1% to 18% by weight, better still from 5% to 16% by weight, better still from 8% to 16% by weight, with respect to the total weight of the composition.

### Fatty Phase

As mentioned above, a composition according to the invention comprises at least one fatty phase. Said fatty phase can contain at least one cosmetic oil. It can additionally contain at least one other fatty substance.

The term “oil” is understood to mean a water-immiscible non-aqueous compound which is liquid at 25° C. and atmospheric pressure (1.013×10⁵ Pa).

The term “immiscible” is understood to mean that the mixing of the same amount of water and of oil, after stirring, does not result in a stable solution comprising only a single phase, under the abovementioned temperature and pressure conditions. Observation is carried out by eye or by means of a phase-contrast microscope, if necessary, on 100 g of mixture obtained after sufficient stirring with a Rayneri blender to produce a vortex within the mixture (by way of indication, from 200 to 1000 rev/min), the resulting mixture being left to stand, in a closed flask, for 24 hours at ambient temperature before observation. Mention may be made, as oils which can be used in the composition of the invention, for example, of:
hydrocarbon oils of vegetable origin, such as liquid triglycerides of fatty acids comprising from 4 to 10 carbon atoms, such as triglycerides of heptanoic or octanoic acids, or also, for example, maize oil, soybean oil, pumpkin oil, grapeseed oil, sesame oil, hazelnut oil, apricot oil, macadamia oil, arara oil, castor oil, avocado oil, caprylic/capric acid triglycerides, such as those sold by Stéarinerie Dubois or those sold under the names Miglyol 810, 812 and 818 by Dynamit Nobel, or shea butter oil;
synthetic esters and ethers, in particular of fatty acids, such as the oils of formulae R₁COOR₂ and R₁OR₂ in which R₁ represents the residue of a fatty acid comprising from 8 to 29 carbon atoms and R₂ represents a branched or unbranched hydrocarbon chain containing from 3 to 30 carbon atoms, such as, for example, Purcellin oil, isononyl isononanoate, cetearyl isononanoate (which is a mixture of C₁₆ and C₁₈ alkyl esters of isononanoic acid), isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate or triisocetyl citrate; fatty alcohol heptanoates, octanoates or decanoates; polyol esters, such as propylene glycol dioctanoate, neopentyl glycol diheptanoate and diethylene glycol diisononanoate; and pentaerythritol esters, such as pentaerythrityl tetraisostearate or dipentaerythrityl pentaisononanoate;
linear or branched hydrocarbons of mineral, vegetable or synthetic origin, such as volatile or non-volatile paraffin oils, and their derivatives, alkanes comprising from 10 to 30 carbon atoms, preferably from 12 to 20 carbon atoms, such as in particular C₁₅₋₁₉ alkanes, such as those sold by Seppic under the name of Emogreen L19, hydrocarbon oils having a branched chain comprising from 10 to 30 carbon atoms, such as isohexadecane, isododecane, isoparaffins and their mixtures, liquid petrolatum, polydecenes, polyisobutenes, squalane, hydrogenated polyisobutenes such as, for example, the Parleam® products sold by Nippon Oil Fats, Panalane H-300 E sold by Amoco, Viseal 20000 sold by Synteal, Rewopal PIB 1000 sold by Witco or also Parleam Lite sold by NOF Corporation. Mention may be made, among the preferred linear or branched hydrocarbons of synthetic origin, of squalane, which is an oil resulting from the hydrogenation of squalene; or
their mixtures.

Preferably, the composition according to the invention comprises at least one oil chosen from linear or branched hydrocarbons of mineral, vegetable or synthetic origin, preferably chosen from alkanes comprising from 10 to 30 carbon atoms, preferably from 12 to 20 carbon atoms, such as in particular C₁₅₋₁₉ alkanes.

The amount of oil in the composition can range, for example, from 0.1% to 15%, preferably from 0.5% to 10%, by weight, with respect to the total weight of the composition.

Preferably, the composition according to the invention is substantially devoid of silicone.—the term “silicone” is understood in particular to mean silicone oils, such as volatile or non-volatile polydimethylsiloxanes (PDMSs) with a linear or cyclic silicone chain, which are liquid or pasty at ambient temperature, in particular cyclopolydimethylsiloxanes (cyclomethicones), such as cyclohexasiloxane; polydimethylsiloxanes comprising alkyl, alkoxy or phenyl groups, which are pendent or at the end of the silicone chain, which groups have from 2 to 24 carbon atoms; or phenylated silicones, such as phenyl trimethicones, phenyl dimethicones, phenyl(trimethylsiloxy)diphenylsiloxanes, diphenyl dimethicones, diphenyl(methyldiphenyl)trisiloxanes, (2-phenylethyl)trimethylsiloxysilicates or also polymethylphenylsiloxanes.

The composition comprises less than 2% by weight of silicone oil(s). According to one embodiment, the composition according to the invention comprises less than 1.0% by weight of silicone oil(s), preferably less than 0.5% by weight of silicone oil(s), with respect to the total weight of the composition, and more preferentially is devoid of silicone oil(s). According to one embodiment, the fatty phase is present in a content of between 5% and 35% by weight, in particular between 8% and 30% by weight and more particularly still between 8% and 12% or between 16% and 28% by weight, with respect to the total weight of the composition.

### Acylamino Acid

The composition according to the invention can also comprise an acylamino acid.

Mention may be made, as acylamino acids, for example, of sodium cocoylglycinate sold by Ajinomoto under the name Amilite® GCS-12, potassium cocoylglycinate sold by Ajinomoto under the names Amilite® GCK-12K (INCI name: Potassium Cocoyl Glycinate) and Amilite® GCK-12H (INCI name: Potassium Cocoyl Glycinate (and) Potassium Cocoate), disodium cocoylglutamate sold by Ajinomoto under the name Amisoft® ECS-22SB, sodium lauroylglutamate sold by Ajinomoto under the name Amisoft® LS 11, sodium lauroylsarcosinate sold by Seppic under the name Oramix™ L 30, sodium stearoylglutamate and disodium stearoylglutamate sold by Ajinomoto under the names Amisoft® HS 11 PF and HS-21P, and sodium cocoylsarcosinate sold by Zschimmer & Schwarz under the name Protelan LS 9011/C.

Mention may also be made of the sodium salt of lauroyl oat amino acids, such as Proteol™ Oat and Proteol™ Oat PF sold by Seppic, or the compound bearing the INCI name Sodium Cocoyl Apple Amino Acids, such as Proteol™ APL from Seppic.

The derivatives of the amino acids can be chosen, for example, from sarcosinates and in particular acylsarcosinates, such as sodium lauroylsarcosinate sold for example under the name Sarkosyl NL 97® by Ciba, sodium myristoylsarcosinate, sold under the name Nikkol Sarcosinate MN® by Nikkol, or sodium palmitoylsarcosinate, sold under the name Nikkol Sarcosinate PN® by Nikkol; alaninates, such as sodium N-lauroyl-N-methylaminopropionate, sold under the name Nikkol Alaninate LN 30® by Nikkol, and triethanolamine N-lauroyl-N-methylalanine, sold under the name Alanone Alta® by Kawaken; aspartates, such as the mixture of triethanolamine N-lauroylaspartate and triethanolamine N-myristoylaspartate, sold under the name Asparack LM-TS2® by Mitsubishi; and citrates.

Mention may also be made of the alkali metal salts of (C₁₀-C₂₂)acylglutamic acids, preferably an alkali metal salt of (C₁₂-C₂₀)acylglutamic acids and for example an alkali metal salt of (C₁₆-C₁₀)acylglutamic acids. The alkali metal salts are, for example, the sodium salts, the potassium salts and the lithium salts, and preferably the sodium salts. It can be a matter in particular of one of the alkali metal salts of stearoylglutamic acid, of lauroylglutamic acid, of a C₁₆ acylglutamic acid, of myristoylglutamic acid, of cocoylglutamic acid or of hydrogenated tallow acylglutamic acid.

Preferably, the acylamino acid is chosen from sodium stearoylglutamate, disodium stearoylglutamate, potassium stearoylglutamate, sodium lauroylglutamate, disodium lauroylglutamate, potassium lauroylglutamate, sodium cocoylglutamate, sodium hydrogenated tallow acylglutamate and their mixtures, and preferably sodium stearoylglutamate.

According to a particular form of the invention, the acylamino acid is sodium stearoylglutamate sold by Ajinomoto under the reference Amisoft® HS 11 PF.

It has in particular been observed that, in the presence of an acylamino acid, such as sodium stearoylglutamate, the composition in the form of an oil-in-water emulsion according to the invention exhibits a microscopic appearance which is finer, corresponding to a fine emulsion, and maintained over time, and also a more limited change in colour over time at a temperature greater than ambient temperature, as illustrated in Example 4.

By way of illustration, mention may also be made, for example, of the disodium stearoylglutamate sold by Ajinomoto under the reference Amisoft® HS 21 P.

Preferably, the acylamino acid is present in concentrations of between 0.01% and 15% by weight, preferably between 0.1% and 3% by weight and more preferentially between 0.2% and 1% by weight, with respect to the total weight of the composition.

### Additives

A composition according to the invention can additionally comprise at least one additive, in particular chosen from the additives detailed below.

Of course, the additive(s) optionally present in a composition according to the invention are chosen so as not to detrimentally affect the properties of the composition, in particular in terms of stability, but also in terms of sensory properties.

Preferably, the additives suitable for the present invention are natural or of natural origin.

### Active Agents

Advantageously, a composition according to the invention can additionally comprise at least one cosmetic active agent.

Mention may be made, as cosmetic active agents, for example, of moisturizing agents, depigmenting agents, desquamating agents, humectants, anti-ageing agents, mattifying agents, healing agents, antibacterial agents, vitamins and their derivatives, antioxidizing compounds, sunscreens and their mixtures.

According to a particular embodiment, the composition according to the invention comprises at least one anti-ageing active agent.

In particular, a composition according to the invention can comprise at least one cosmetic active agent chosen from hyaluronic acid or one of its derivatives exhibiting a molecular weight of less than 50 000 Da, in particular sodium hyaluronate exhibiting a molecular weight of less than 50 000 Da.

### Hyaluronic Acid

In the context of the present invention, the term “hyaluronic acid or one of its derivatives” covers in particular the base unit of hyaluronic acid of following formula (8):

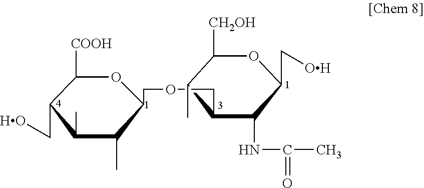

This is the smallest fraction of hyaluronic acid, comprising a disaccharide dimer, namely D-glucuronic acid and N-acetylglucosamine.

The term “hyaluronic acid or one of its derivatives exhibiting a molecular weight of less than 50 000 daltons” comprises, in the context of the present invention, the linear polymer comprising the polymeric unit described above, according to a sequence with alternating β(1,4) and β(1,3) glycosidic bonds, having a low molecular weight (MW) which can vary between 380 and 50 000 daltons. This molecular weight mainly depends on the source from which the hyaluronic acid is obtained and/or on the preparation methods.

The term “hyaluronic acid or one of its derivatives” also comprises, in the context of the present invention, the salts of hyaluronic acid and in particular the alkali metal salts, such as the sodium salt and the potassium salt, in particular sodium hyaluronate.

In the natural state, hyaluronic acid is present in pericellular gels, in the ground substance of connective tissues of vertebrate organs, such as the dermis and epithelial tissues, and in particular in the epidermis, in the articular synovial fluid, in the vitreous humour, in the human umbilical cord and in the crista galli.

Thus, the term “hyaluronic acid or one of its derivatives” comprises all of the fractions or subunits of hyaluronic acid exhibiting a molecular weight in particular within the molecular weight range recalled above.

By way of illustration of the various hyaluronic acid fractions, reference may be made to the document “Hyaluronan Fragments: An Information-Rich System”, R. Stern et al., European Journal of Cell Biology, 58 (2006), 699-715, which reviews the listed biological activities of hyaluronic acid as a function of its molecular weight.

Finally, the term “hyaluronic acid or one of its derivatives” also comprises esters of hyaluronic acid, in particular those in which all or some of the carboxylic groups of the acid functions are esterified with oxyethylenated alkyls or alcohols comprising from 1 to 20 carbon atoms, in particular with a degree of substitution on the D-glucuronic acid of the hyaluronic acid varying from 0.5% to 50%.

Mention may in particular be made of the methyl, ethyl, n-propyl, n-pentyl, benzyl and dodecyl esters of hyaluronic acid. Such esters have in particular been described in D. Campoccia et al., “Semisynthetic Resorbable Materials from Hyaluronan Esterification”, Biomaterials, 19 (1998), 2101-2127.

The molecular weights indicated above are also valid for the hyaluronic acid esters.

Hyaluronic acid can in particular be provided by Contipro under the trade name HyActive (MW: 10 to 150 kDa), by Soliance (Givaudan) under the name Renovhyal LO (50 kDa), by Bloomage BioTechnology under the name Hybloom™ Low Molecular Weight Sodium Hyaluronate (HA-TLM), by Bioland under the name Oligo HA (MW: 6100 Da) or also by Vam Farmacos Metica under the name D Factor (MW: 380 Da).

In one embodiment, sodium hyaluronate exhibiting a molecular weight of less than 50 000 Da is used.

In particular, hyaluronic acid or one of its derivatives is present in the composition according to the present invention in a content of between 0.05% and 10% by weight, preferably between 0.5% and 3.0% by weight, more preferentially between 0.01% and 0.5% by weight, with respect to the total weight of the composition.

In a particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one polymer or one copolymer of 2-acrylamido-2-methylpropanesulfonic acid (AMPS) which is optionally crosslinked and/or neutralized, and
at least one acylamino acid,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least two hydrophilic gelling agents,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid, preferably comprising at least hydrogenated lecithin,
at least one hydrophilic gelling agent comprising at least one polymer or one copolymer of 2-acrylamido-2-methylpropanesulfonic acid (AMPS) which is optionally crosslinked and/or neutralized, preferably comprising at least one crosslinked and neutralized homopolymer of 2-acrylamido-2-methylpropanesulfonic acid, and
at least one additional hydrophilic gelling agent, preferably comprising at least one polysaccharide, more preferentially still comprising at least xanthan gum,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one polymer or one copolymer of 2-acrylamido-2-methylpropanesulfonic acid (AMPS) which is optionally crosslinked and/or neutralized, and
at least one additional hydrophilic gelling agent,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one polymer or one copolymer of 2-acrylamido-2-methylpropanesulfonic acid (AMPS) which is optionally crosslinked and/or neutralized, and
at least one additional hydrophilic gelling agent comprising at least one polysaccharide,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one polymer or one copolymer of 2-acrylamido-2-methylpropanesulfonic acid (AMPS) which is optionally crosslinked and/or neutralized,
at least one additional hydrophilic gelling agent,
at least one acylamino acid, and
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one polymer or one copolymer of 2-acrylamido-2-methylpropanesulfonic acid (AMPS) which is optionally crosslinked and/or neutralized,
at least one additional hydrophilic gelling agent comprising at least one polysaccharide,
at least one acylamino acid, and
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one crosslinked and neutralized homopolymer of 2-acrylamido-2-methylpropanesulfonic acid, and
at least one acylamino acid comprising at least sodium stearoylglutamate,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one crosslinked and neutralized homopolymer of 2-acrylamido-2-methylpropanesulfonic acid, and
at least one additional hydrophilic gelling agent,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one crosslinked and neutralized homopolymer of 2-acrylamido-2-methylpropanesulfonic acid, and
at least one additional hydrophilic gelling agent comprising at least xanthan gum,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one crosslinked and neutralized homopolymer of 2-acrylamido-2-methylpropanesulfonic acid,
at least one additional hydrophilic gelling agent,
at least one acylamino acid comprising at least sodium stearoylglutamate, and
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least one phospholipid,
at least one hydrophilic gelling agent comprising at least one crosslinked and neutralized homopolymer of 2-acrylamido-2-methylpropanesulfonic acid,
at least one additional hydrophilic gelling agent comprising at least xanthan gum,
at least one acylamino acid comprising at least sodium stearoylglutamate, and
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least hydrogenated lecithin,
at least one hydrophilic gelling agent comprising at least one crosslinked and neutralized homopolymer of 2-acrylamido-2-methylpropanesulfonic acid, and
at least one acylamino acid comprising at least sodium stearoylglutamate,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least hydrogenated lecithin,
at least one hydrophilic gelling agent comprising at least one crosslinked and neutralized homopolymer of 2-acrylamido-2-methylpropanesulfonic acid, and
at least one additional hydrophilic gelling agent,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least hydrogenated lecithin,
at least one hydrophilic gelling agent comprising at least one crosslinked and neutralized homopolymer of 2-acrylamido-2-methylpropanesulfonic acid, and
at least one additional hydrophilic gelling agent, preferably comprising at least one polysaccharide, more preferentially comprising at least xanthan gum,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least hydrogenated lecithin,
at least one hydrophilic gelling agent comprising at least one crosslinked and neutralized homopolymer of 2-acrylamido-2-methylpropanesulfonic acid, and
at least one additional hydrophilic gelling agent comprising at least xanthan gum,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least hydrogenated lecithin,
at least one hydrophilic gelling agent comprising at least one crosslinked and neutralized homopolymer of 2-acrylamido-2-methylpropanesulfonic acid,
at least one additional hydrophilic gelling agent, and
at least one acylamino acid comprising at least sodium stearoylglutamate,
   characterized in that the composition comprises less than 2.0% by weight of silicone oil, with respect to the total weight of the composition.

In another particular embodiment, the composition in the form of an oil-in-water emulsion, which is in particular cosmetic, especially for making up and/or caring for keratin materials, comprises:
at least one fatty phase,
at least one emulsifier comprising at least hydrogenated lecithin,
at least one hydrophilic gelling agent comprising at least one crosslinked and neutralized homopolymer of 2-acrylamido-2-methylpropanesulfonic acid,
at least one additional hydrophilic gelling agent comprising at least xanthan gum, and
at least one acylamino acid comprising at least sodium stearoylglutamate,
   characterized in that the composition comprises less than 2.0% by weight of silicone oil, with respect to the total weight of the composition.

Concrete but in no way limiting examples illustrating the invention will now be given.

In the examples, the temperature is ambient temperature (20° C.) and is expressed in degrees Celsius, unless otherwise indicated, and the pressure is atmospheric pressure, unless otherwise indicated.

In the examples, the amounts of the ingredients of the compositions are “expressed as % of starting material”, more specifically as % by weight of starting material, with respect to the total weight of the composition.

### EXAMPLES

### Equipment and Methods

Measurement of the Stability The stability of the compositions was evaluated according to the following protocol:
1) The following first checks are carried out on a test composition a minimum of 16 hours after manufacture:
   organoleptic checks (appearance, colour, smell);
   a pH measurement (at 25° C.): measurement taken using a pH meter;
   a viscosity measurement (at 25° C.): measurement taken using a viscometer, for example Rheomat RM 100 Plus from Lamy Rheology (equipped with a thermostatic bath), marking of the values at 30 seconds and 10 minutes;
   centrifugation carried out for 1 hour at 900 g, followed by verification of the absence of any sign of destabilization (such as salting out, sedimentation, creaming, and the like); and
   microscopic observation of a withdrawn sample of the composition crushed between a slide and a cover glass in non-polarized light and in polarized light, with a magnification of ×100 (times 100), the characterization of the observation being carried out in particular by the evaluation of the fineness of the emulsion, the mesh of the centre, the uniformity of the emulsion, the edges and the anisotropy or isotropy.
2) The test composition is subsequently stored for 2 months at different temperatures, in particular at 4° C., at ambient temperature, noted as AT, at 37° C. and at 45° C. The temperature can be regulated, for example using an oven, in particular for a temperature equal to 37° C. or 45° C., for example of Bio brand concept of Firlabo, or using a refrigerator, in particular for a temperature equal to 4° C.
3) Checks are carried out after storage for 1 month and 2 months under the conditions described above. In particular, the checks carried out are as follows:
   the macroscopic appearance: checking of the visual appearance and the texture, including the consistency, and confirmation of the absence of any sign of instability, for example a setting or a softening, the appearance of grains, a salting out, a creaming, a coalescence, the formation of a film, the display of mottling, of exudate;
   the colour: evaluation of the potential change in colour of the samples in comparison with the 4° C. sample, referred to as “control” sample;
   the smell: evaluation of the potential change in smell of the samples in comparison with the 4° C. sample, referred to as “control” sample;
   the microscopic appearance: evaluation of the potential change in the microscopic appearance in non-polarized light and polarized light (magnification ×100);
   the pH, only for the samples at 2 months at AT and at 2 months at 45° C.; and
   the viscosity, only for the samples only at 2 months at AT and at 2 months at 45° C.

The closer the checks on the composition after storage for 1 month and/or 2 months are to the checks carried out at a time of greater than 16 hours from manufacture, the more the composition can be regarded as remaining stable. In particular, if the composition remains smooth and homogeneous, without salting out or phase separation, with an acceptable change in colour, smell, viscosity, pH and microscopic appearance, then it the composition regarded as being stable.

### Protocol for Preparation of the Compositions

The compositions of Examples 1 to 4 are prepared according to the following protocol:
The ingredients of phase A are mixed and the mixture is heated to 70° C.;
The ingredients of phase C are mixed and the mixture is heated to 75° C.;
The ingredient B is added to phase A and the pellets are allowed to melt for 5 min under moderate stirring with paddles;
During this time, the ingredient D is added to phase C and the temperature of the C+D mixture is maintained at 70° C.;
Subsequently, the C+D mixture is added to the A+B mixture, the temperatures of the phases being identical and equal to 70° C., and the mixture is left for 5 min under moderate stirring with paddles;
The emulsion is subsequently formed under a rotor-stator for 10 min and then cooled under moderate stirring with paddles;
The ingredient E and then, if appropriate, the ingredient F and then the ingredient G are added under a rotor-stator at a temperature <30° C., with stirring for 5 min after each addition.

### Example 1

The compositions F1 and F2 according to the invention are obtained according to the preparation protocol described above and are detailed in the following Table 1.

TABLE 1

| | Ingredient | | | |
|---|---|---|---|---|
| | (expressed as % of | Commercial | | |
| Phase | starting material) | reference | F1 | F2 |
| A | Water | | q.s. | q.s. |
| | Glycerol | Glicenat GC K MB | 10.00 | 10.00 |
| | | (Oxiteno) | | |
| A | Hydrophilic active agents | | 1.35 | — |
| A | Solvent (for active agent) | | 0.25 | — |
| A | Dyes | | 0.001 | — |
| A | Chelating agent | | 0.15 | 0.15 |
| | Preservative | | 0.20 | — |
| | pH adjusters | | q.s. | q.s. |
| | | | for | for |
| | | | pH 5.3 | pH 5.3 |
| B | C12-C16 Alcohols (and) | Biophilic ™ H MB | 3.50 | 2.50 |
| | Palmitic Acid (and) | (Lucas Meyer | | |
| | Hydrogenated Lecithin* | Cosmetics) | | |
| C | Behenyl alcohol* | Lanette 22 (BASF) | 2.00 | 1.50 |
| | Anionic surfactant* | — | 1.00 | 1.00 |
| | Emollients* | — | 14.00 | 7.25 |
| | Preservatives | | 0.30 | 0.80 |
| | Ammonium | Hostacerin AMPS ® | 1.00 | 1.00 |
| | Polyacryloyldimethyl | (Clariant) | | |
| | Taurate | | | |
| | Xanthan gum | Keltrol CG (CP Kelco) | 0.10 | 0.10 |
| | Sodium hyaluronate | Renovhyal LO | 0.10 | — |
| | | (Soliance) | | |
| D | Shea butter | Lipex 102 | 5.00 | 4.00 |
| | (Butyrospermum Parkii | (AarhusKarlshamn) | | |
| | (Shea) Butter)* | | | |
| D | Antioxidant | | 0.25 | 0.10 |
| E | Lipophilic active agents | | — | 1.00 |
| E | Fragrance | | 0.20 | — |
| F | Denat. Alcohol | Ethyl Alcohol 99D | 4.80 | — |
| | | (Aroma Holly Limited) | | |

*Ingredients constituting the fatty phase

The technical effects relating to the sensoriality and to the effect on the skin were demonstrated via consumer tests for these compositions F1 and F2 according to the invention, as set out in Example 5 below.

### Example 2

The composition F3 according to the invention is obtained according to the preparation protocol described above and is detailed in the following Table 2.

TABLE 2

| | Ingredient | | |
|---|---|---|---|
| | (expressed as % of | Commercial | |
| Phase | starting material) | reference | F3 |
| A | Water | | q.s. |
| | Glycerol | Glicenat GC K MB | 15.00 |
| | | (Oxiteno) | |
| | Hydrophilic active agents | | 4.40 |
| | Chelating agent | | 0.15 |
| | Preservative | | 0.20 |
| | pH adjusters | | q.s for |
| | | | pH 5.3 |
| B | C12-C16 Alcohols (and) | Biophilic ™ H MB | 3.50 |
| | Palmitic Acid (and) | (Lucas Meyer | |
| | Hydrogenated Lecithin* | Cosmetics) | |
| C | Behenyl alcohol* | Lanette 22 (BASF) | 2.00 |
| | Anionic surfactant* | — | 1.00 |
| | Emollient* | — | 16.50 |
| | Preservative | | 0.30 |
| | Ammonium | Hostacerin AMPS ® | 1.00 |
| | Polyacryloyldimethyl | (Clariant) | |
| | Taurate | | |
| | Xanthan gum | Keltrol CG (CP Kelco) | 0.10 |
| | Sodium hyaluronate | Renovhyal LO | 0.10 |
| | | (Soliance) | |
| D | Shea butter (Butyrospermum | Lipex 102 | 8.00 |
| | Parkii (Shea) Butter)* | (AarhusKarlshamn) | |
| | Antioxidant | | 0.10 |
| E | Denat. Alcohol | Ethyl Alcohol 99D | 4.80 |
| | | (Aroma Holly Limited) | |

*Ingredients constituting the fatty phase

Results: After measurement of the stability according to the protocol described above, the check on the macroscopic appearance of the composition F3 is in accordance; in particular, no sign of instability is perceptible, whatever the storage conditions at 4° C., AT, 37° C. and 45° C.

Conclusion: the composition F3, comprising the combination of ammonium polyacryldimethyltaurate, xanthan gum and hydrogenated lecithin, exhibits good stability.

### Example 3

A comparison is made between two compositions (F5 and F6) according to the invention, one comprising xanthan gum as additional gelling agent (composition 15) and the other not comprising xanthan gum (composition F6). These two compositions are obtained according to the preparation protocol described above and are detailed in the following Table 3.

TABLE 3

| | Ingredient | | | |
|---|---|---|---|---|
| | (expressed as % of | Commercial | | |
| Phase | starting material) | reference | F5 | F6 |
| A | Water | | q.s. | q.s. |
| | Glycerol | Glicenat GC K MB | 15.00 | 15.00 |
| | | (Oxiteno) | | |
| | Hydrophilic active agents | | 4.40 | 4.40 |
| | Chelating agent | | 0.15 | 0.15 |
| | Preservative | | 0.20 | 0.20 |
| | pH adjusters | | q.s. | q.s. |
| | | | for | for |
| | | | pH 5.3 | pH 5.3 |
| B | C12-C16 Alcohols (and) | Biophilic ™ H MB | 3.50 | 3.50 |
| | Palmitic Acid (and) | (Lucas Meyer | | |
| | Hydrogenated Lecithin* | Cosmetics) | | |
| C | Behenyl alcohol* | Lanette 22 (BASF) | 2.00 | 2.00 |
| | Anionic surfactant* | | 1.00 | 1.00 |
| | Emollient* | | 16.50 | 16.50 |
| | Preservative* | | 0.30 | 0.30 |
| | Ammonium | Hostacerin AMPS ® | 1.00 | 1.00 |
| | Polyacryloyldimethyl | (Clariant) | | |
| | Taurate | | | |
| | Xanthan gum | Keltrol CG (CP | 0.10 | — |
| | | Kelco) | | |
| | Sodium hyaluronate | Renovhyal LO | 0.50 | 0.50 |
| | | (Soliance) | | |
| D | Shea butter | Lipex 102 | 8.00 | 8.00 |
| | (Butyrospermum Parkii | (AarhusKarlshamn) | | |
| | (Shea) Butter)* | | | |
| | Antioxidant | | 0.10 | 0.10 |
| E | Denat. Alcohol | Ethyl Alcohol 99D | 4.80 | 4.80 |
| | | (Aroma Holly Limited) | | |

*Ingredients constituting the fatty phase

### Results:

Observation with a microscope, in particular under magnification ×100 and in non-polarized light, shows that the composition F5 appears finer and more uniform than the composition F6. In particular, the size of the globules observed with a microscope is less than 5 μm for the composition F5, whereas the size of the globules observed with a microscope is greater than 5 μm for the composition F6.

Conclusion: a composition also comprising an additional gelling agent, such as xanthan gum, exhibits a finer and more uniform microscopic appearance than a composition devoid of this additional gelling agent, which may afford an advantageous additional benefit, for example in terms of stability. In particular, a finer and more uniform microscopic appearance of a composition can be associated with a more advantageous long-term stability, in particular a stability which is more robust over time.

### Example 4

A comparison is made between two compositions (F7 and F8) according to the invention, one comprising sodium stearoylglutamate as acylamino acid (composition F7) and the other being devoid of sodium stearoylglutamate (composition F8). These two compositions are obtained according to the preparation protocol described above and are detailed in the following Table 4.

TABLE 4

| | Ingredient | | | |
|---|---|---|---|---|
| | (expressed as % of | Commercial | | |
| Phase | starting material) | reference | F7 | F8 |
| A | Water | | q.s. | q.s. |
| | Glycerol | Glicenat GC K MB | 10.00 | 10.00 |
| | | (Oxiteno) | | |
| | Hydrophilic active agents | | 4.40 | 4.40 |
| | Chelating agent | | 0.15 | 0.15 |
| | Preservative | | 0.20 | 0.20 |
| | pH adjusters | | q.s. | q.s. |
| | | | for | for |
| | | | pH 5.3 | pH 5.3 |
| B | C12-C16 Alcohols (and) | Biophilic ™ H MB | 3.50 | 3.50 |
| | Palmitic Acid (and) | (Lucas Meyer | | |
| | Hydrogenated Lecithin* | Cosmetics) | | |
| C | Behenyl alcohol* | Lanette 22 (BASF) | 2.00 | 2.00 |
| | Anionic surfactant* | | 1.00 | 1.00 |
| | Sodium Stearoyl | Amisoft HS 11 PF | — | 0.40 |
| | Glutamate | (Ajinomoto) | | |
| | Emollient* | | 16.50 | 16.50 |
| | Preservative | | 0.30 | 0.30 |
| | Ammonium | Hostacerin AMPS ® | 1.00 | 1.00 |
| | Polyacryloyldimethyl | (Clariant) | | |
| | Taurate | | | |
| | Xanthan gum | Keltrol CG (CP Kelco) | 0.10 | 0.10 |
| | Sodium hyaluronate | Renovhyal LO | 0.10 | 0.10 |
| | | (Soliance) | | |
| D | Shea butter | Lipex 102 | 8.00 | 8.00 |
| | (Butyrospermum Parkii | (AarhusKarlshamn) | | |
| | (Shea) Butter)* | | | |
| | Antioxidant | | 0.10 | 0.10 |
| E | Charge | | 1.00 | 1.00 |
| F | Fragrance | | 0.30 | — |
| G | Denat. Alcohol | Ethyl Alcohol 99D | 4.80 | 4.80 |
| | | (Aroma Holly Limited) | | |

*Ingredients constituting the fatty phase

### Results:

During the measurement of the stability of the compositions F7 and F8, a more marked difference in change in colour between the two compositions is observed after 2 months at 45° C. In particular, after 2 months at 45° C., the composition F8 comprising sodium stearoylglutamate undergoes a less marked change in colour, compared with the composition F7 devoid of sodium stearoylglutamate.

Moreover, with regard to the microscopic appearance, the composition F8 comprising sodium stearoylglutamate is a finer oil-in-water emulsion, compared to the composition F7 devoid of sodium stearoylglutamate. In addition, the microscopic appearance of the composition F8 comprising sodium stearoylglutamate does not change after 2 months at 45° C.

Conclusion: a composition additionally comprising an acylamino acid, such as sodium stearoylglutamate, exhibits a limited change in colour after 2 months at 45° C. and corresponds to a finer emulsion, in comparison with a composition devoid of this acylamino acid, which may afford an advantageous additional benefit, in particular with regard to the stability.

### Example 5—Consumer Tests

The technical effects relating to the sensoriality and to the effect on the skin have been demonstrated via consumer tests.

For example, the composition F1 of Example 1 was evaluated in the anti-ageing axis by 120 targeted consumers after 4 weeks of use, in replacement of their usual product. The consumers are women aged from 50 to 70 years who are regular users of an anti-ageing day cream.

The results obtained show that the composition F1 according to the invention exhibits the following advantages:
it has a pleasant texture;
it leaves an instantaneous feeling of freshness on the skin;
it immediately nourishes the skin and gives it a feeling of comfort and of hydration which lasts throughout the day, without a greasy effect;
it allows the skin to breathe; and
it is gentle and beneficial to the skin.

In addition, the composition F2 of Example 1 was evaluated in the hydration axis by 180 targeted consumers after 2 weeks of use, in replacement of their usual product. The consumers are women aged from 25 to 75 years having normal to very dry skin, with signs of dryness, in particular of rough, scaly, taut, itchy, irritated or reactive skin, and are regular users of a moisturizing day cream.

The results obtained show that the composition F2 according to the invention exhibits the following advantages:
it has a pleasant texture, which spreads well over the skin, with a good rate of penetration;
it allows the skin to breathe, makes it more supple, softer and smoother to the touch;
it immediately gives the skin a feeling of comfort and of hydration, which lasts throughout the day;
it prevents signs of dryness throughout the day; and
it improves the quality of the skin and gives it radiance.

1. A composition in the form of an oil-in-water emulsion, which is cosmetic, for making up and/or caring for keratin materials, comprising:
at least one fatty phase,
at least one emulsifier comprising at least one phospholipid, and
at least one hydrophilic gelling agent,
wherein the composition comprises less than 2.0% by weight of silicone oil, with respect to the total weight of the composition.

2. The composition according to claim 1, wherein the phospholipid is chosen from:
natural phospholipids,
chemically or enzymatically modified phospholipids, and
synthetic phospholipids.

3. The composition according to claim 1, wherein the phospholipid is present in an amount of between 0.01% and 3% by weight with respect to the total weight of the composition.

4. The composition according to claim 1, wherein the phospholipid is present in a mixture with fatty alcohols, and/or with a fatty acid.

5. The composition according to claim 4, wherein the hydrophilic gelling agent is chosen from:
carboxyvinyl polymers;
polymers and copolymers of 2-acrylamido-2-methylpropanesulfonic acid, which are optionally crosslinked and/or neutralized;
heterogeneous polyholosides;
glyceryl acrylate polymers;
polysaccharides;
clays; and
their mixtures.

6. The composition according to claim 1, wherein the hydrophilic gelling agent is chosen from polymers and copolymers of 2-acrylamido-2-methylpropanesulfonic acid, including the crosslinked and neutralized homopolymer of 2-acrylamido-2-methylpropanesulfonic acid, which can be present in a content of between 0.1% and 10% by weight with respect to the total weight of the composition.

7. The composition according to claim 1, wherein the composition comprises at least one hydrophilic gelling agent chosen from polymers and copolymers of 2-acrylamido-2-methylpropanesulfonic acid, and at least one additional gelling agent chosen from polysaccharides, said additional gelling agent being present in a content of between 0.01% and 2% by weight with respect to the total weight of the composition.

8. The composition according to claim 1, wherein the fatty phase is present in a content of between 5% and 35% by weight, with respect to the total weight of the composition.

9. The composition according to claim 1, comprising less than 1.0% by weight of silicone oil(s) with respect to the total weight of the composition.

10. The composition according to claim 1, wherein the composition additionally comprises an anti-ageing active agent.

11. The composition according to claim 10, wherein the anti-ageing active agent is chosen from hyaluronic acid or one of its derivatives exhibiting a molecular weight of less than 50 000 Da.

12. The composition according to claim 1, wherein the composition additionally comprises an acylamino acid, in a content of between 0.01% and 15% by weight with respect to the total weight of the composition.

13. The composition according to claim 1, wherein the aqueous phase is present in a content of between 65% and 95% by weight with respect to the total weight of the composition.

14. A cosmetic process for caring for keratin materials, including the skin, comprising at least a stage of application, to said keratin materials, of a composition as defined according to claim 1.

15. The composition according to claim 2, wherein the natural phospholipids are selected from the group consisting of egg or soya lecithin, and sphingomyelin, wherein the chemically or enzymatically modified phospholipids is hydrogenated lecithin, and wherein the synthetic phospholipids is selected from the group consisting of dipalmitoylphosphatidylcholine, and hydrogenated lecithin.

16. The composition according to claim 4, wherein the fatty alcohols is an alcohol having from 12 to 16 carbon atoms, and the fatty acid is palmitic acid.

17. The composition according to claim 5, wherein the polymers and copolymers of 2-acrylamido-2-methylpropanesulfonic acid, that are optionally crosslinked and/or neutralized, are selected from the group consisting of: (a) poly(2-acrylamido-2-methylpropanesulfonic acid) polymers, and the homopolymers, polyAMPS, which are crosslinked and at least 90% neutralized, and (b) AMPS copolymers, including (i) copolymers of AMPS and of acrylamide, for example crosslinked anionic copolymers of acrylamide and of AMPS, (ii) copolymers derived from 2-acrylamido-2-methylpropanesulfonic acid which are or are not crosslinked, comprising at least one hydrophobic group, (iii) copolymers of AMPS and of vinylpyrrolidone or of vinylformamide, (iv) copolymers of AMPS and of sodium acrylate, (v) copolymers of AMPS and of hydroxyethyl acrylate, and (vi) copolymers of AMPS and of biopolymer.

18. The composition according to claim 7, wherein the polymers and copolymers of 2-acrylamido-2-methylpropanesulfonic acid is a crosslinked and neutralized homopolymer of 2-acrylamido-2-methylpropanesulfonic acid, and the gelling agent is either xanthan gum or carrageenan.

19. The composition according to claim 9, wherein the composition is devoid of silicone oil(s).

20. The composition according to claim 11, wherein the anti-ageing active agent is sodium hyaluronate exhibiting a molecular weight of less than 50 000 Da.
